(19) United States
(12) Patent Application Publication
(10) Pub. No.: US 2007/0260304 A1
(43) Pub. Date: Nov. 8, 2007

(54) Bifurcated stent with minimally circumferentially projected side branch

(76) Inventors: Daniel Gregorich, St. Louis Park, MN (US); Michael P. Meyer, Richfield, MN (US)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 11/415,527

(22) Filed: May 2, 2006

(51) Int. Cl. A61F 2/06

(52) U.S. Cl. 623/1.35

(57) ABSTRACT

The present invention is directed toward a bifurcation stent comprising a plurality of interconnected expansion columns, wherein the plurality of interconnected expansion columns are longitudinally spaced from the proximal end of the stent to the distal end of the stent and wherein the plurality of interconnected expansion columns define a plurality of cells. The inventive stent further comprises a framework defining a side branch cell. The framework is designed to minimize the circumferential space the side branch structure consumes. A plurality of expansion columns are connected to the framework. When the side branch is extended and expanded, the connected expansion columns form the wall of the side branch and the framework expands to conform to the shape of a targeted bodily vessel.

### FIELD OF THE INVENTION

In some embodiments this invention relates to implantable medical devices, their manufacture, and methods of use. Some embodiments are directed to delivery systems, such as catheter systems of all types, which are utilized in the delivery of such devices.

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

Stents may be arranged for bifurcations and may include outwardly deployable side branch structure. However, because expansion characteristics of the side branch structure are often different than portions of the stent, stent designs that would be sufficiently flexible to traverse a tortuous anatomy in an unexpanded state sometimes would not provide adequate vessel support in the expanded state.

There remains a need for stent patterns that provide proper scaffolding support and drug delivery in the expanded state, while also allowing for crimpability and for flexibility and deliverability in the unexpanded state.

Prior to delivery a stent or stents may be retained on a portion of the delivery catheter by crimping the stent onto the catheter, retaining the stent in a reduced state about the catheter with a removable sheath, sleeve, sock or other member or members, or by any of a variety of retaining mechanisms or methods. Some examples of stent retaining mechanisms are described in U.S. Pat. No. 5,534,007; U.S. Pat. No. 5,681,345; U.S. Pat. No. 5,788,707; U.S. Pat. No. 5,968,069; U.S. Pat. No. 6,066,155; U.S. Pat. No. 6,096,045; U.S. Pat. No. 6,221,097; U.S. Pat. No. 6,331,186; U.S. Pat. No. 6,342,066; U.S. Pat. No. 6,350,277; U.S. Pat. No. 6,443,980; and U.S. Pat. No. 6,478,814.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention is directed toward a bifurcation stent comprising a plurality of interconnected expansion columns, wherein the plurality of interconnected expansion columns are longitudinally spaced from the proximal end of the stent to the distal end of the stent and wherein the plurality of interconnected expansion columns define a plurality of cells. The inventive stent further comprises a framework defining a side branch cell. The framework is designed to minimize the circumferential space the side branch structure consumes and therefore maximize the circumferential portions of the stent main body which are associated with the side branch structure. A plurality of expansion columns which make up a portion of the stent main body are connected to the framework. When the side branch is extended and expanded, portions of the connected expansion columns form the wall of the side branch and the framework expands to conform to the shape of a targeted bodily vessel.

In some embodiments, when the stent is in its unexpanded state, the framework, or side branch cell, is positioned within the circumferential plane of the main body of the stent and defines an opening. In maximizing the circumferential portions of the stent main body which are connected to the framework, the framework is designed such that undulating portions of the framework extend circumferentially between adjacent expansion columns. The framework takes up a minimal amount of space in the circumferential direction to allow the contralateral expansion columns to crimp to a comparable minimum diameter as the main branch geometry.

In some embodiments, the stent has a contracted state and an expanded state. The stent, when in its contracted state includes a primary tubular body defining a lumen having a proximal end and a distal end and an axis extending through the proximal and distal ends. The primary tubular body further comprises a plurality of continuous expansion columns and a plurality of discontinuous expansion columns, wherein the plurality of continuous expansion columns and the plurality of discontinuous expansion columns are interconnected. The plurality of discontinuous expansion columns are between a plurality of the continuous expansion columns positioned proximally to the plurality of the discontinuous expansion columns and a plurality of the continuous expansion columns positioned distally to the plurality of the discontinuous expansion columns. When the stent is in its expanded state, the stent further comprises a secondary tubular body defining a lumen and has an axis extending therethrough, wherein the lumen of the secondary tubular body is in fluid communication with the lumen of the primary tubular body and the axis of the secondary tubular body is at an oblique angle relative to the axis of the primary tubular body. The secondary tubular body comprises a side wall positioned around the axis of the secondary tubular body and terminating end, wherein the side wall is between the terminating end and the primary tubular body and wherein the side wall is formed by the plurality of discontinuous expansion columns.

In at least one embodiment, the invention is directed to a stent having a proximal end and a distal end. The stent further comprises a plurality of interconnected strut members defining a plurality of cells. A portion of the interconnected strut members comprise a side branch framework defining a side branch cell, wherein the side branch cell is shaped differently than other cells of the stent. The interconnected strut members further define a plurality of serpentine bands and a plurality of connector struts. Adjacent serpentine bands are connected by at least one connector strut and at least two of the serpentine bands are connected to the side branch framework.

In at least one other embodiment, a stent may be made according to a flat pattern comprising a plurality of interconnected strut members defining a plurality of cells. A portion of the interconnected strut members comprise a side branch framework defining a side branch cell, the side branch cell being shaped differently than other cells of the stent. The interconnected strut members further define a plurality of serpentine bands and a plurality of connector struts. Adjacent serpentine bands are connected by at least one connector strut. A first serpentine band has a first band axis, and at least a portion of the first band axis extends perpendicular to a stent lengthwise axis. A second serpentine band has a second band axis, and at least a portion of the second band axis extends circumferentially about a portion of the stent. The first and second serpentine bands are both connected to the side branch framework.

In at least one other embodiment, a stent comprises a plurality of interconnected strut members defining a plurality of cells. A portion of the interconnected strut members comprise a side branch framework defining a side branch cell, the side branch cell being shaped differently than other cells of the stent. The interconnected strut members further define a plurality of serpentine bands and a plurality of connector struts. Adjacent serpentine bands are connected by at least one connector strut. Each serpentine band comprises a plurality of proximal peaks and distal valleys. A first serpentine band is connected to the side branch framework and a second serpentine band is connected to the side branch framework. A third serpentine band and a fourth serpentine band are also connected to the side branch framework.

In at least some embodiments, the serpentine bands that are connected to the framework have an increased amplitude and/or frequency along their individual axes relative to the serpentine bands that are not connected to the framework. In some embodiments, the amplitude and/or frequency of the serpentine bands that are connected to the framework may vary within each of the individual bands and may vary between the individual bands.

In at least some embodiments, a stent comprises a plurality of interconnected expansion columns, wherein the plurality of interconnected expansion columns are longitudinally spaced from the proximal end of the stent to the distal end of the stent and wherein the plurality of interconnected expansion columns define a plurality of cells. The stent further comprises a framework defining a side branch cell, the framework having a proximal end and a distal end and a length defined by the distance between the proximal and distal ends of the framework and being positioned between the proximal end and the distal end of the stent. The plurality of interconnected expansion columns include a first expansion column, wherein first expansion column is circumferentially oriented relative to the axis of the stent and wherein the first end and second end of the first expansion column are opposingly connected to the framework. The plurality of interconnected columns further include a second expansion column, wherein the second expansion column is circumferentially oriented relative to the axis of the stent and is longitudinally adjacent to the first expansion column. The first end and second end of the second expansion column are opposingly connected to the framework and the distance between the first end and the second of the first expansion column and the distance between the first end and the second end of the second expansion column are equal to or less than ⅓ of the length of the framework.

In at least some embodiments of the present invention, the first ends of the first and second expansion columns are linearly aligned parallel with the axis of the stent. In at least some embodiments, the second ends of the first and second expansion columns are also linearly aligned parallel with the axis of the stent.

In at least some embodiments, the stent further comprises a third expansion column longitudinally adjacent to the first or second expansion column. The first end and second end of the third expansion column are opposingly connected to the framework and the distance between the first end and the second of the third expansion column is equal to or less than ⅓ of the length of the framework. The first ends of the first, second and third expansion columns may be linearly aligned parallel with the axis of the stent. Still further, the second ends of the first, second and third expansion columns may also be linearly aligned parallel with the axis of the stent.

In at least some embodiments, the stent further comprises a fourth expansion column, wherein the first and second expansion columns are longitudinally between the third and fourth expansion columns. The first end and second end of the fourth expansion column are opposingly connected to the framework and the distance between the first end and the second of the fourth expansion column is equal to or less than ¼ of the length of the framework. The first ends of the first, second, third and fourth expansion columns are linearly aligned parallel with the axis of the stent. Still further, the second ends of the first, second third and fourth expansion columns may be linearly aligned parallel with the axis of the stent.

In at least some embodiments, the framework has an axis extending through the proximal and distal ends of the framework and comprises a plurality of finger-like projections circumferentially extending between adjacent expansion columns from the axis of the framework around the axis of the stent. Some of the finger-like projections may extend in a first direction and the remaining projections extend in a direction opposite to that of the first direction.

In some embodiments of the invention, when it is in its expanded state, the stent comprises a main body and a side branch extending therefrom. The main body is a tubular structure having a plurality of interconnected expansion columns, wherein the plurality of interconnected expansion columns are longitudinally spaced from the proximal end of the main body to the distal end of the main body and wherein the plurality of interconnected expansion columns define a plurality of cells. The plurality of interconnected expansion columns comprises a first expansion column and a second expansion column. The stent further comprises a side branch extending radially from the main body between the proximal and distal ends of the main body. The side branch comprises an expanded framework, the expanded framework being the terminating end of the side branch and having a ring shaped configuration. The side branch further comprises a side branch wall positioned between the main body and the expanded framework. The first and second ends of the first and second expansion columns are connected to the expanded framework and form at least part of the side branch wall. In some embodiments, two or more additional expansion columns may be connected to the expanded framework, further forming the side branch wall.

The present invention also further includes methods of delivering the disclosed inventive stents to a bifurcation site in a bodily vessel.

These and other embodiments that characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 shows a top view of a flat pattern for an embodiment of a stent.

FIG. 2 shows a perspective view of an embodiment of the inventive stent.

FIG. 3 shows a top view of a flat pattern for another embodiment of a stent.

FIG. 4 shows a perspective view of an embodiment of the inventive stent.

FIG. 4A shows a blown up top view of a portion of the stent of FIG. 4.

FIG. 5 shows a perspective view of an embodiment of the inventive stent.

FIG. 5A shows a top view of the stent of FIG. 5.

FIG. 6 is an end view of the stent of FIG. 5.

FIGS. 7A-C show side views of an embodiment of the invention mounted on a balloon illustrating expansion from a contracted state to an expanded state.

FIGS. 8A-B show side views of an embodiment of the invention mounted on a balloon illustrating expansion from a contracted state to an expanded state.

FIG. 9 shows a top view of an individual serpentine band.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, or substituted for, elements depicted in another figure as desired.

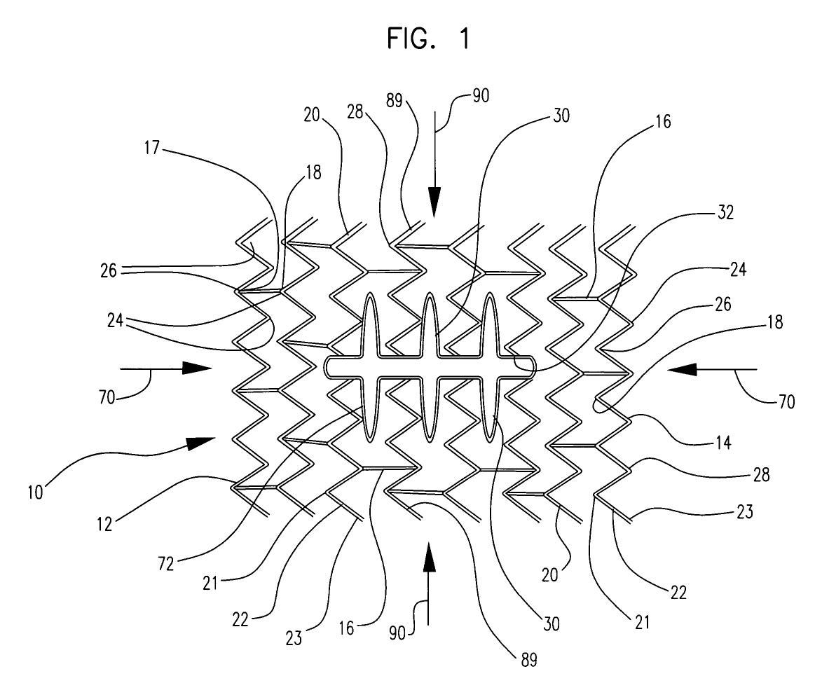

FIG. 1 shows an embodiment of a flat pattern for a stent 10. The stent 10 may have a proximal end 12 and a distal end 14, and may comprise a plurality of serpentine bands 20. Each serpentine band 20 may comprise a plurality of struts 22, each strut 22 having a first end 21 and a second end 23. Circumferentially adjacent struts 22 within a serpentine band 20 may be connected by turns 28. Turns 28 located on a serpentine band 20 may comprise alternating peaks 24 and valleys 26.

Serpentine bands 20 that are adjacent to one another along the length of the stent 10 may be connected by at least one connector strut 16. A connector strut 16 may span between turns 28 of adjacent serpentine bands 20. For example, a first end 17 of a connector strut 16 may connect to a valley 26 of one serpentine band 20, and a second end 18 of the connector strut 16 may connect to a peak 24 of an adjacent serpentine band 20. In some embodiments, a connector strut 16 may connect to any portion of a serpentine band 20, such as a strut 22, a valley 26 or a peak 24. A connector strut 16 may have any suitable shape and may be straight along its length, or in some embodiments may have curvature, bends, inflection points, etc. It should also be understood that the adjacent serpentine bands 20 may be aligned with one another in various ways. For example peaks 24 may face valleys 26 or peaks 24 may face peaks 24 or the patterns may be staggered.

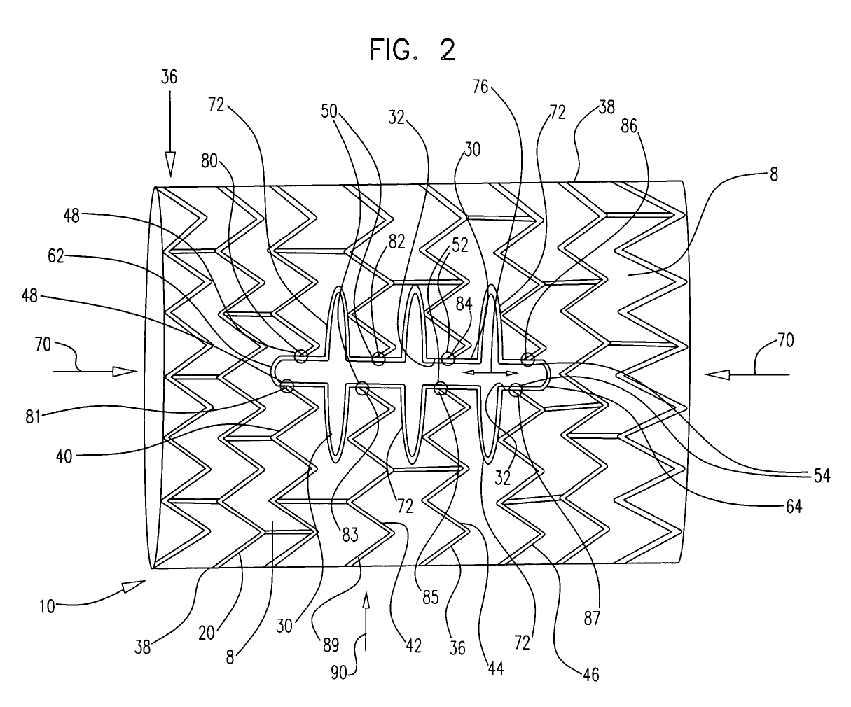

When the flat pattern for the stent 10 takes on a tubular configuration, as shown in FIG. 2, each of the serpentine bands 20 forms a circumferential expansion column 36. The plurality of expansion columns 36 forms the main body or primary tubular body 38 of the stent 10. The expansion columns 36 may have a radially contracted state and may be expandable to an expanded state. This capability allows the main body 38 of the stent 10 to be contracted and expanded. Methods of forming tubular stents and contracting and expanding them are well known to those skilled in the art of stent design and manufacture.

The interconnected expansion columns 36 may define a stent wall portion and may further define a plurality of cells 8. Each cell 8 may comprise an aperture or void in the stent wall portion. The cells 8 may be uniform in shape or may vary depending on the positioning of the connector struts 16.

The stent 10 may further comprise a side branch cell or opening 30 that is different than other cells 8 of the stent 10. As shown in FIG. 2, the side branch cell 30 has a framework 32 that defines the side branch cell 30. A framework is a continuous piece of material that defines a closed inner space. In this particular embodiment, the framework 32 takes on the particular configuration shown in FIGS. 1-2. However, other shapes are contemplated.

In the particular embodiment shown, the side branch cell 30, and therefore the framework 32, has a first end 62 and a second end 64 and has a length 31 that is longitudinally oriented relative to the axis 70 of the stent 10. As can be seen, the length of the framework and the side branch cell 30 longitudinally extend across at least three serpentine bands 20 and at least three expansion columns 36. Finger-like projections 72 of the side branch cell 30 extend circumferentially from the axis 76 of the side branch cell 30 between adjacent expansion columns 36. In the embodiment shown, there are six finger-like projections 72. It should be understood that the invention contemplates fewer or more finger-like projections 72.

In the particular embodiment shown in FIG. 2, a plurality of expansion columns (36) 40, 42, 44, 46, are connected to the framework 32 at pairing points 48, 50, 52 and 54, respectively. These expansion columns (40, 42, 44, 46) are considered to discontinuous expansion columns because they are not circumferentially continuous around the axis 70 of the stent 10 as compared to the remaining expansion columns 36. They are continuous, however, from their first ends (80, 82, 84, 86) to their second ends (81, 83, 85, 87). More or fewer expansion columns 36 may be connected to the framework 32, but there are least two.

Each serpentine band 20 which make up the plurality of expansion columns 40, 42, 44, 46, which are connected to the framework 32 has a first end (80, 82, 84, 86) and a second end (81, 83, 85, 87). The first (80, 82, 84, 86) and second (81, 83, 85, 87) ends of the plurality of serpentine bands 20 which form expansion columns 40, 42, 44, 46, are shown to be connected to the framework 32 at pairing points 48, 50, 52 and 54, respectively.

As can be seen, in some embodiments, the first end (80, 82, 84, 86) of each of the serpentine bands 20, which is connected to the framework 32, is in close proximity to its respective second end (81, 83, 85, 87), relative to the length 31 of the side branch cell 30. In some embodiments, the ratio of the distance between the first (80, 82, 84, 86) and second ends (81, 83, 85, 87) to the length 31 of the framework is less than 1:3. In some of the embodiment, the ratio is less than 1:4 and in other embodiments the ratio is less than 1:5. These ratios may apply to two or more pairs of the first and second ends.

As can be seen in FIGS. 1-2, in some embodiments of the invention, the first ends (80, 82, 84, 86) of the connected serpentine bands 20 are substantially linearly aligned with one another and the second ends (81, 83, 85, 87) of the connected serpentine bands 20 are substantially linearly aligned with one another. In the embodiments shown, both the first ends (80, 82, 84, 86) and the second ends (81, 83, 85, 87) are substantially aligned parallel with the axis 70 of the stent 10.

In some embodiments of the invention, when the stent 10 is viewed as a flat pattern, as shown in FIG. 1, the longitudinal axes 90 of the various serpentine bands 20 which are connected to the framework 32, such as serpentine band 89, may all be oriented at a perpendicular angle along the length of the serpentine bands 20 relative to the axis 70 of the stent 10.

In some embodiments of the invention, the framework 32 is, relative to the stent axis 70, longitudinally between a plurality of expansion columns 36 beyond one end 62 of the framework 32 and a plurality of expansion columns 36 beyond the other end 64 of the framework 32.

The side branch cell 30 may take on any shape that provides a narrow opening and allows for expansion columns 36 which are connected to the framework 32 which defines the opening 30 to be circumferentially substantially complete as compared to expansion columns 36 within the stent 10 which are not connected to the framework 32 of the opening 30.

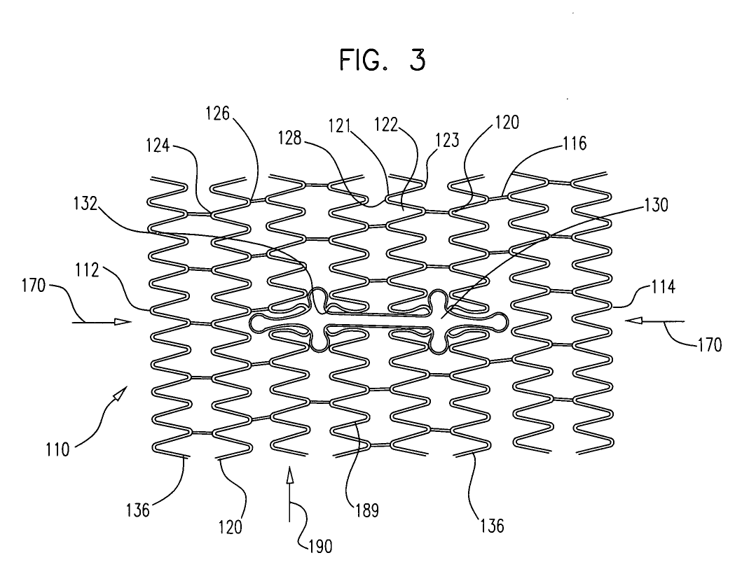

FIG. 3 shows an embodiment of a flat pattern for a stent 110. Stent 110 is the same as stent 10, except that it illustrates a different side branch cell or opening 130 and framework 132 design. As with stent 10, the stent 110 may have a proximal end 112 and a distal end 114, and may comprise a plurality of serpentine bands 120. Each serpentine band 120 may comprise a plurality of struts 122, each strut 122 having a first end 121 and a second end 123. Circumferentially adjacent struts 122 within a serpentine band 120 may be connected by turns 128. Turns 128 located on a serpentine band 120 may comprise alternating peaks 124 and valleys 126.

Serpentine bands 120, which are adjacent to one another along the length of the stent 110, may be connected by at least one connector strut 116. A connector strut 116 may span between turns 128 of adjacent serpentine bands 120. A connector strut 16 may have any suitable shape and may be straight along its length, or in some embodiments may have curvature, bends, inflection points, etc. It should also be understood that the adjacent serpentine bands 120 may be aligned with one another in various ways. For example peaks 124 may face valleys 126 or peaks 124 may face peaks 124 or the patterns may be staggered.

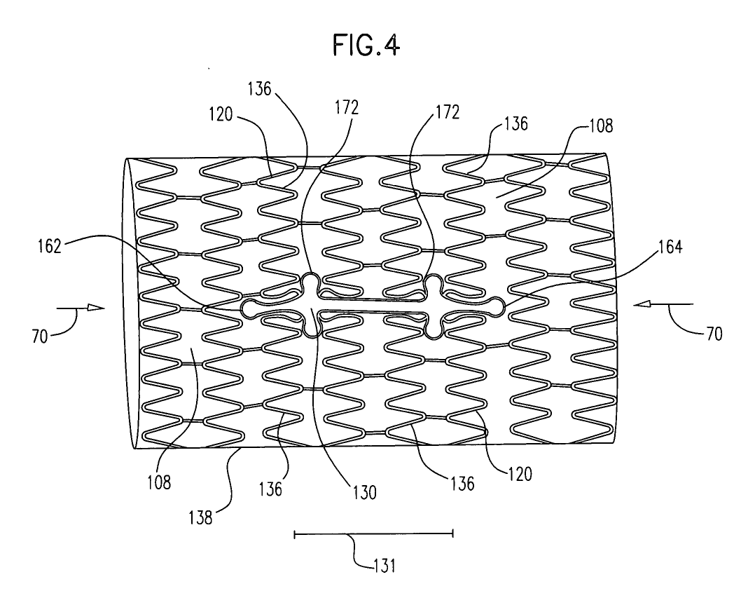

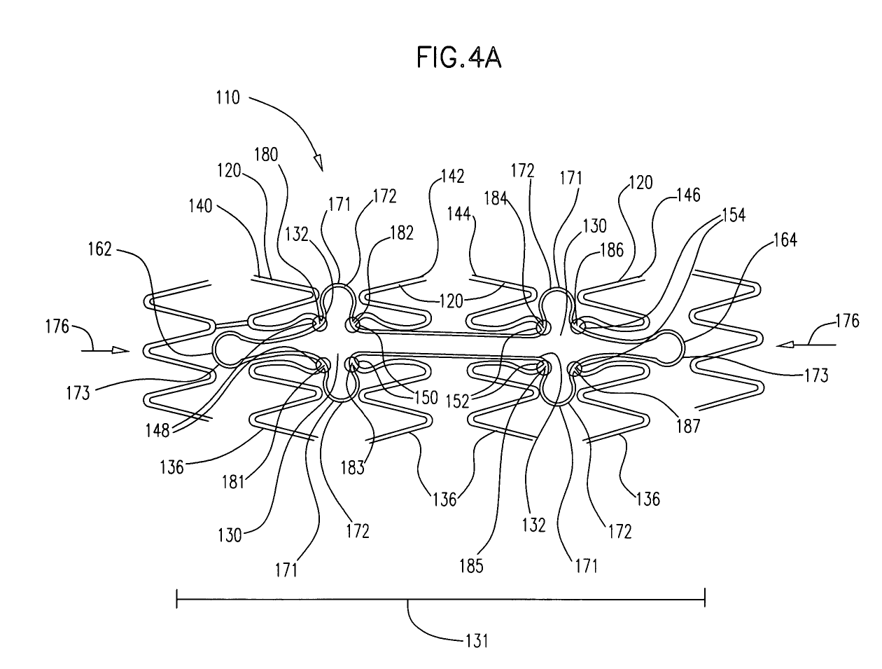

When the flat pattern for the stent 110 takes on a tubular configuration, as shown in FIG. 4, each of the serpentine bands 120 forms a circumferential expansion column 136. The plurality of expansion columns 136 forms the main body 138 of the stent 110. The expansion columns 136 may have a radially contracted state and may be expandable to an expanded state and visa versa. This capability allows the main body 138 of the stent 110 to be contracted and expanded. Methods of forming tubular stents and contracting and expanding them are well known to those skilled in the art of stent design and manufacture.

The interconnected expansion columns 136 may define a stent wall portion and may further define a plurality of cells 108. Each cell 108 may comprise an aperture or void in the stent wall portion. The cells 108 may be uniform in shape or may vary depending on the positioning of the connector struts 116.

The stent 110 further comprises a side branch cell or opening 130 that is different than other cells 108 of the stent 110 and of a different design than side branch cell 30 of stent 10. As shown in FIG. 4 and partial blown up FIG. 4A showing the side branch cell 130, the side branch cell 130 has a framework 132 which defines the side branch cell 130.

In the particular embodiment shown in FIGS. 4 and 4A, the side branch cell 130, and therefore the framework 132, have a first end 162 and a second end 164 and has a length 131, which is longitudinally oriented relative to the axis 170 of the stent 110. As can be seen, the length of the framework and the side branch cell 30 longitudinally extend across at least three serpentine bands 20 and at least three expansion columns 36. Finger-like projections 172 of the side branch cell 130 extend circumferentially from the axis 176 of the side branch cell 130 between adjacent expansion columns 136. In the embodiment shown, there are four finger-like projections 72. It should be understood that the invention contemplates fewer or more finger-like projections 72.

In some embodiments, the projections 172 extending between adjacent expansion columns 136 terminate with a bulbous configuration 171. The ends 162, 164 of the framework 132 may also terminate with a bulbous configuration 173. In some embodiments, the extending projections 72, 172, of the framework 32, 132, are longitudinally separated from the ends 62, 64, 162, 164, of the framework 32, 132, by at least one expansion column 36, 136.

In the particular embodiment shown in FIG. 4A, a plurality of expansion columns (136) 140, 142, 144, 146, are connected to the framework 132 at pairing points 148, 150, 152 and 154, respectively. These expansion columns (140, 142, 144, 146) are considered to discontinuous expansion columns because they are not circumferentially continuous around the axis 170 of the stent 110 as compared to the remaining expansion columns 136. More or fewer expansion columns 136 may be connected to the framework 132, but there are least two. Each serpentine band 120 which make up the plurality of expansion columns 140, 142, 144, 146, which are connected to the framework 132 has a first end (180, 182, 184, 186) and a second end (181, 183, 185, 187). The first (180, 182, 184, 186) and second (181, 183, 185, 187) ends of the plurality of serpentine bands 120 which form expansion columns 140, 142, 144, 146, are shown to be connected to the framework 132 at pairing points 148, 150, 152 and 154, respectively.

As can be seen, in some embodiments, the first end (180, 182, 184, 186) of each of the serpentine bands 120, which is connected to the framework 132, is in close proximity to its respective second end, relative to the length 131 of the side branch cell 130. As mentioned above, in some embodiments, the ratio of the distance between the first (180, 182, 184, 186) and second ends (181, 183, 185, 187) to the length 131 of the framework is less than 1:3. In some of the embodiment, the ratio is less than 1:4 and in other embodiments the ratio is less than 1:5. These ratios may apply to two or more pairs of the first and second ends.

As can be seen in FIGS. 4-4A, in some embodiments of the invention, the first ends (180, 182, 184, 186) of the connected serpentine bands 120 are substantially linearly aligned with one another and the second ends (181, 183, 185, 187) of the connected serpentine bands 120 are substantially linearly aligned with one another. In the embodiments shown, both the first ends (180, 182, 184, 186) and the second ends (181, 183, 185, 187) are substantially aligned parallel with the axis 170 of the stent 110.

In some embodiments of the invention, when the stent 110 is viewed as a flat pattern, as shown in FIG. 31, the longitudinal axes 190 of the various serpentine bands 120 which are connected to the framework 132, such as serpentine band 189, may all be oriented at a perpendicular angle along the length of the serpentine bands 120 relative to the axis 170 of the stent 110.

In some embodiments of the invention, the framework 132 is, relative to the stent axis 170, longitudinally between a plurality of expansion columns 136 beyond one end 162 of the framework 132 and a plurality of expansion columns 136 beyond the other end 164 of the framework 132. For the embodiments shown in FIGS. 1-2 and 3-4A, there are two expansion columns 36, 136, on either side of the frameworks 32, 132, shown. It should be understood that the number of expansion columns 36, 136, which sandwich the framework 32, 132, may vary.

The side branch cell 130 may take on any shape that provides a narrow opening and allows for expansion columns 136 which are connected to the framework 132, which defines the side branch cell 130 to be circumferentially substantially complete as compared to expansion columns 136 within the stent 110 which are not connected to the framework 132 of the opening 130.

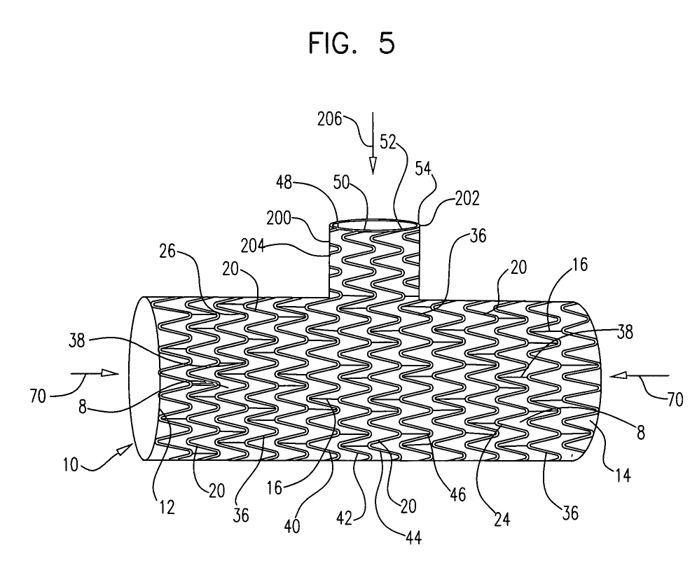

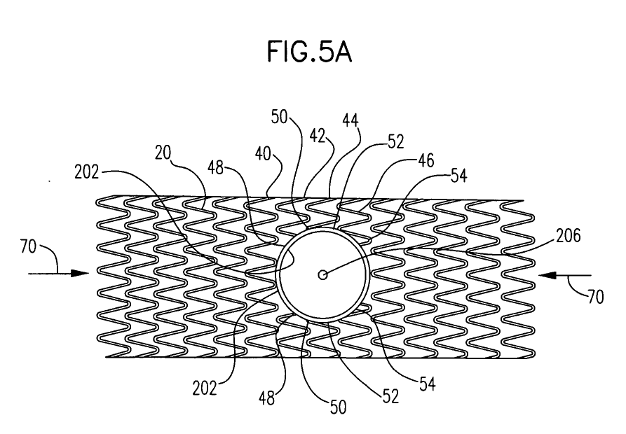

FIG. 5 illustrates the stents of the invention in their expanded state. Although the illustration represents expanded states of the stents 10, 110, shown in FIGS. 1-2 and 3-4A, as well as those stents having different side branch cell configurations which are contemplated by the invention, for elements which have already been identified, only reference numerals from FIGS. 1-2 are used. For example, instead of identifying serpentine bands with 20 and 120, they are only identified with numeral 20.

In some embodiments, as shown in FIG. 5, the main body 38 of the stent 10 may be radially expanded with methods well known in the art. An expanded side branch or secondary tubular body 200 is also shown extending from the main body 38 at some point between the ends 12, 14, of the stent 10.

As can be seen in FIG. 5, in some embodiments, expansion columns 40, 42, 44 and 46 and the expanded framework 202 form the side branch. As mentioned above, the number of expansion columns that are connected to the framework 32 (expanded framework 202) may vary. The connected expansion columns 40, 42, 44, 46, project outward from the main body 38 forming the side wall 204 of the side branch 200 allowing for greater side branch coverage and support. As can be seen, the expansion columns 40, 42, 44, 46, that make up the side branch 200 have the same geometry as the expansion columns 36 of the main body 38 of the stent 10.

FIG. 5A is an illustration of the stent of FIG. 5 looking down on side branch 200 of the stent 10. As can be seen, during the extension and expansion of the side branch 200, the framework 32 is expanded to form an expanded framework 202. The expanded framework 202 has a generally circular or oval shape to conform to the bodily side branch vessel targeted. It should be understood that the expanded framework 202 may be merely generally circular and that it is in a halo-type position over the main body 38 of the stent 10. In some embodiments, the axis 206 of the side branch 200 may be perpendicular to the axis 70 of the main body 38 of the stent or it may be at any oblique angle (between 0 and 180 degrees) relative to the axis 70 of the main body 38. The angle may be dictated by the orientation of the bifurcated bodily vessel which is targeted.

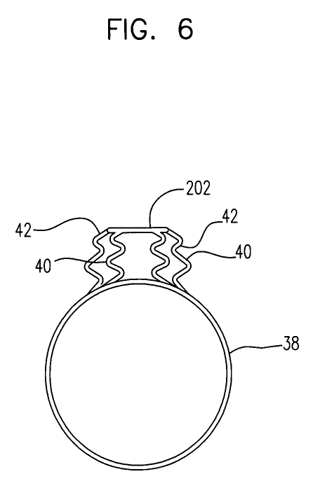

FIG. 6 is an illustration of the stent of FIG. 5 looking down the longitudinal axis 70 of the main body 38 of the stent 10. As can be seen, expansion column 40 spirals upward to its connection with the framework 32.

The side branch geometry can be deployed into the side branch 200 by using a second side branch balloon or by utilizing a mechanism attached to the side branch lumen to pull the side branch support geometry into the side branch 200 as the lumen is advanced. Some embodiments of delivery catheters that may be suitable for delivering and deploying stents as described herein are disclosed in U.S. Pat. No. 6,835,203 and US Published Application No. 20050060027, the entire disclosures of which are hereby incorporated herein in their entireties.

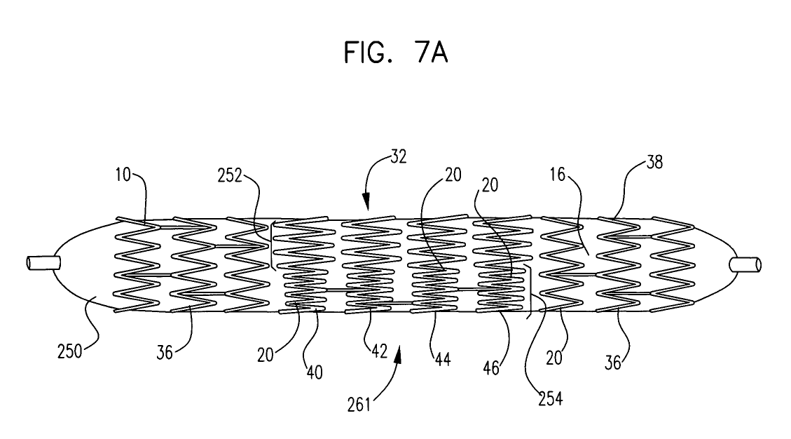

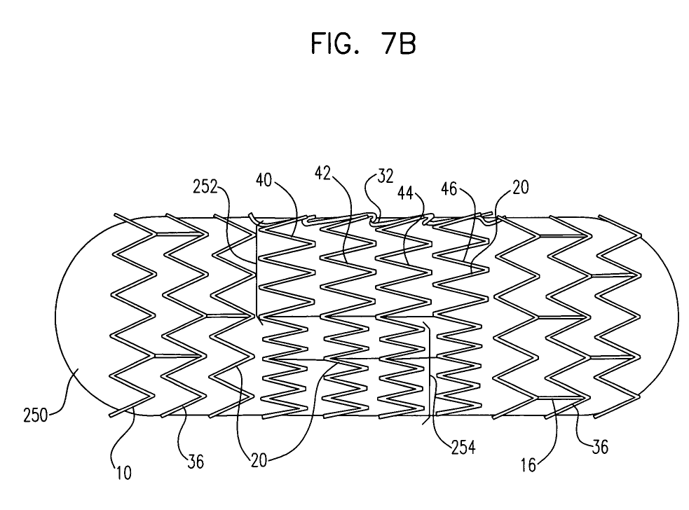

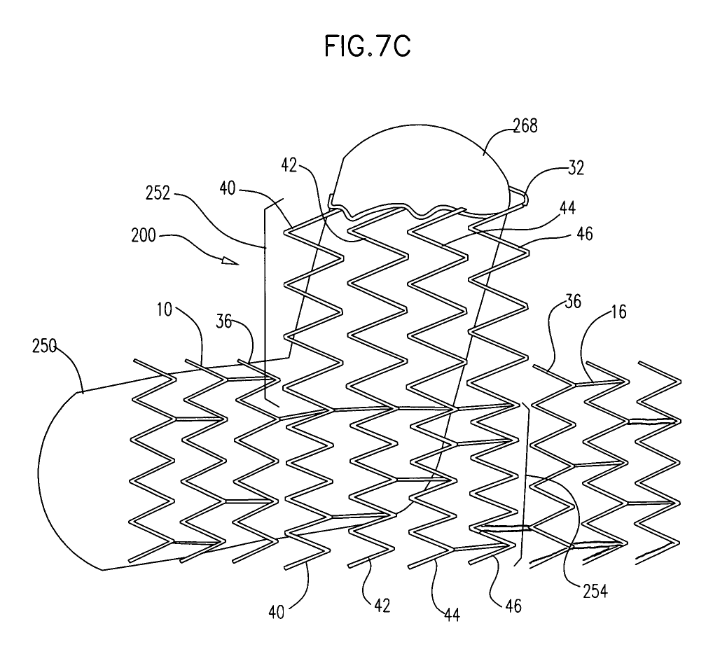

In expansion of the secondary tubular body 200, a balloon from underneath expands upwards, projecting the side branch structure outward. FIGS. 7A-C and 8A-B are illustrative representations of examples of the expansion of the stents of the present invention from a loaded position to an expanded condition.

FIGS. 7A-C show a stepwise expansion of the stent 10 using a balloon 250. It should be understood that the balloon 250 is typically mounted on the distal end of a delivery catheter. Such constructions are well known in the art. In FIG. 7A, the stent 10 is shown loaded and crimped onto the balloon 250 in its contracted condition. The view is from the side of the stent 10 such that the framework 32 is on the upper side of the balloon 250 cannot be clearly seen. Also, as can be seen, the expansion columns (40, 42, 44, 46) connected to the framework 32 comprise serpentine bands 20 that have a higher frequency and/or amplitude of struts 22 than those expansion columns 36 that are not connected to the framework 32 and are on the proximal and distal sides of the framework 32. In other words, the band length, meaning the length of the serpentine band 20 if it were stretched out to form a straight line, of the expansion columns 40, 42, 44 and 46 is greater than that of the expansion columns that are not connected to the framework 32. This is to accommodate the extension of the side branch 200, as shown in FIG. 7C, since the expansion columns (40, 42, 44, 46) that form the side branch 200 have to extend further than the other expansion columns 36. This higher frequency and/or amplitude of the struts 22 may make up a portion of the expansion columns (40, 42, 44, 46) that form the side branch 200 or may make up the entire expansion columns. The extent of increase of higher frequency and/or amplitude may vary within each expansion column (40, 42, 44, 46) and/or between the expansion column (40, 42, 44, 46). Also, since the serpentine bands 20 of expansion columns 40 and 46 may have to extend further to accommodate their connecting positions on the framework 32 when the side branch 200 is extended, they may have in increased amplitude and/or frequency relative to the other expansion columns 42, 44, that are connected to the framework. As can be seen in FIG. 6, these expansion columns 40, 46, have to spiral up to the side of the framework and travel a greater distance that expansion columns 42 and 44.

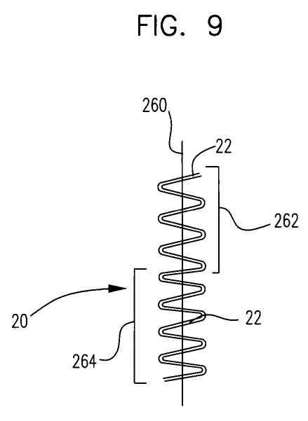

A serpentine band 20 in flat form is shown in FIG. 9. What is meant by a higher amplitude is that the struts extend laterally from the centerline 260 to a greater extent, as shown by portion 262 relative to portion 264. What is meant by a higher frequency is that the number of struts along a particular segment of the centerline 260 is greater, as shown by portion 264 relative to portion 262.

In some embodiments, as shown in FIGS. 7A-B, the portions 252 of the expansion columns (40, 42, 44, 46) that form the side branch 200 have a greater strut 20 amplitude and/or a lower strut 20 frequency than portions 254 of the expansion columns (40, 42, 44, 46) that are generally contralateral 260 to the framework 32. Overall, the expansion columns expansion columns (40, 42, 44, 46) that are connected to the framework 32 may have a greater amplitude and/or strut 22 frequency than those expansion columns 36 that are not connected to the framework 32. The degree of either may vary along the length of the serpentine bands 20 which make up the expansion columns 40, 42, 44 and 46.

FIG. 7B illustrates the balloon 250 and main body 38 of the stent 10 in their expanded condition after the balloon 250 has been inflated. As can be seen, the expansion columns 36 which are not connected to the framework 32 are extended to a greater extent than the expansion columns (40, 42, 44, 46) connected to the framework 32. The expansion columns (40, 42, 44, 46) have more band 20 material to accommodate the further extension of the side branch 200.

After expansion of the main body 38 of the stent, the distal end 268 of the balloon 250 is then introduced into the center of the framework 32 and extended and expanded up through the framework 32. This action extends the expansion columns (40, 42, 44, 46) connected to the framework 32 and expands the framework 32 to form the side branch 200.

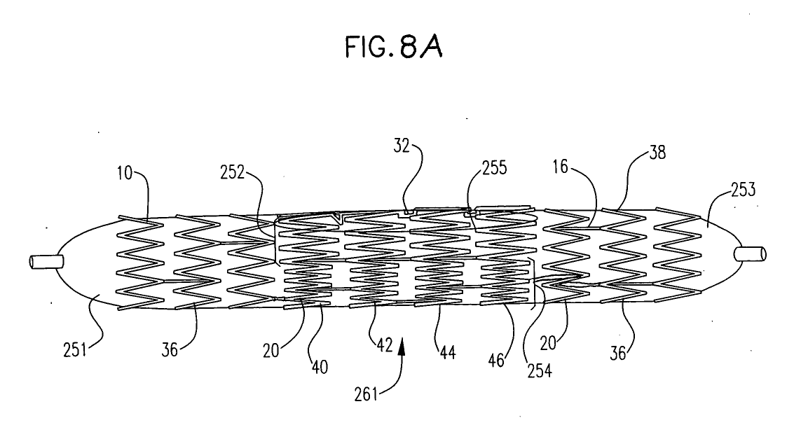

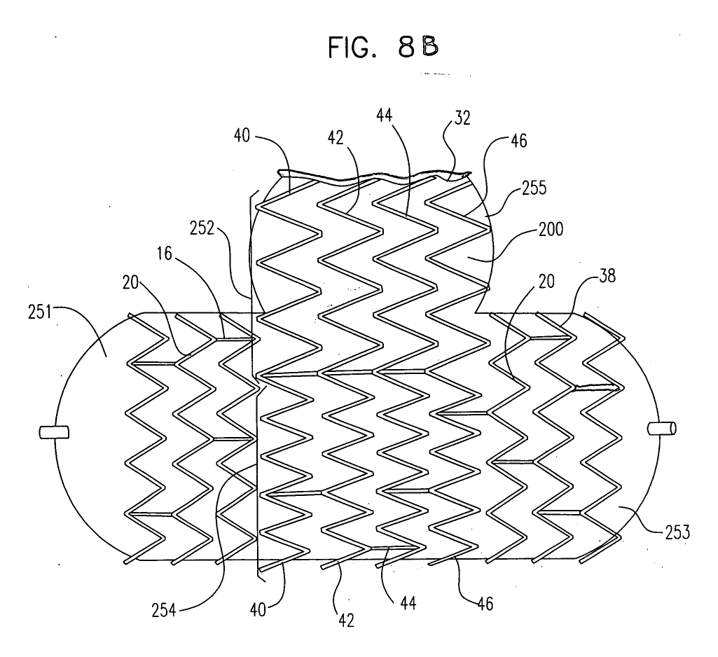

FIG. 8A illustrate the expansion of the stent 10 with an alternative balloon 251 type. In this illustration, the balloon 251 has a main body 253 and a side branch portion 255 which expands and extends the side branch 200 of the stent 10. The side branch portion 255 of the balloon 251 may be separately inflatable. FIG. 8A shows the balloon 251 and stent 10 in their contracted condition. As shown in FIG. 8B, upon expansion of the balloon 251, the main body 38 of the stent is expanded. Simultaneously or after the expansion of the main body 253 of the balloon, the side branch portion 255 of the balloon 251 is expanded, extending and expanding the side branch 200 of the stent 10.

The invention is further directed to methods of manufacturing a stent according to the designs disclosed herein. The invention is further directed to methods of delivering and expanding a stent as described herein.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials that are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

The present invention may be incorporated into both of the two basic types of catheters used in combination with a guide wire, commonly referred to as over-the-wire (OTW) catheters and rapid-exchange (RX) catheters. The construction and use of both over-the-wire and rapid-exchange catheters are well known in the art.

The present invention may also be incorporated into bifurcated assemblies. Examples of such systems are shown and described in U.S. patent application Ser. No. 10/375,689, filed Feb. 27, 2003 and U.S. patent application Ser. No. 10/657,472, filed Sep. 8, 2003 both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; U.S. patent application Ser. No. 10/757,646, filed Jan. 13, 2004 and entitled Bifurcated Stent Delivery System; U.S. Patent Publication 20040088007; and U.S. patent application Ser. No. 10/784,337, filed Feb. 23, 2004 and entitled Apparatus and Method for Crimping a Stent Assembly; the entire content of each of which are incorporated herein by reference.

Embodiments of the present invention can be incorporated into those shown and described in the various references cited above. Likewise, embodiments of the inventions shown and described therein can be incorporated herein.

In at least one embodiment, a method of delivering a stent to a bifurcation comprises the steps of a) advancing any of the stent assemblies above disposed about a catheter along two guidewires and b) deploying the stent at the bifurcation site.

In at least one embodiment, a method of delivering a stent to a bifurcation comprises the steps of a) advancing any of the stent assemblies above disposed about a catheter along two guidewires, the catheter having a catheter shaft and catheter balloon, the stent assembly disposed about the catheter balloon and b) deploying the stent assembly at the bifurcation site by expanding the catheter balloon. In at least one embodiment, expansion of the catheter balloon acts on the stent assembly to expand the stent assembly such that the stent assembly expands to an expanded state.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the stent is configured to include one or more mechanisms for the delivery of a therapeutic agent. Often the agent will be in the form of a coating or other layer (or layers) of material placed on a surface region of the stent and is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A stent having a contracted state, wherein the stent is expandable from the contracted state to an expanded state, the stent comprising:
a primary tubular body defining a lumen having a proximal end and a distal end and an axis extending through the proximal and distal ends, the primary tubular body further comprising:
a plurality of continuous expansion columns and a plurality of discontinuous expansion columns, wherein the plurality of continuous expansion columns and the plurality of discontinuous expansion columns are interconnected and circumferentially positioned around the axis of the primary tubular body and are longitudinally spaced from the proximal end of the primary tubular body to the distal end of the primary tubular body and wherein the plurality of discontinuous expansion columns are between a plurality of the continuous expansion columns positioned proximally to the plurality of the discontinuous expansion columns and a plurality of the continuous expansion columns positioned distally to the plurality of the discontinuous expansion columns,
wherein, when the stent is in its expanded state, the stent further comprises:
a secondary tubular body defining a lumen and having an axis extending therethrough, wherein the lumen of the secondary tubular body is in fluid communication with the lumen of the primary tubular body and the axis of the secondary tubular body is at an oblique angle relative to the axis of the primary tubular body, the secondary tubular body comprising a side wall positioned around the axis of the secondary tubular body and terminating end, wherein the side wall is between the terminating end and the primary tubular body and wherein the side wall is formed by the plurality of discontinuous expansion columns.

2. The stent of claim 1, the stent further comprising a framework defining a side branch cell and being positioned within the circumferential plane of the primary tubular body, wherein the plurality of discontinuous expansion columns are directly connected to the framework and wherein, when the stent is in its expanded state, the framework is expanded to form the terminating end of the secondary tubular body.

3. The stent of claim 2, the plurality interconnected continuous expansion columns and discontinuous expansion columns defining a plurality of cells, wherein the side branch cell has a different shape than the plurality of cells.

4. The stent of claim 3, the framework comprising a spatial center point and plurality of undulations forming a plurality of peaks, wherein the plurality of peaks extend away from the spatial center point and circumferentially around the axis of the primary tubular body, such that they are longitudinally position between adjacent discontinuous and continuous expansion columns.

5. The stent of claim 4, wherein there are at least two peaks circumferentially extending in one direction and at least two separate peaks extending circumferentially in an opposite direction.

6. The stent of claim 4, the framework having a proximal end and a distal end and a length defined by the distance between the proximal and distal ends of the framework and being positioned between the proximal end and the distal end of the primary tubular body, wherein the length longitudinally extends across at least four discontinuous and/or continuous expansion columns.

7. The stent of claim 3, wherein the plurality interconnected continuous expansion columns and discontinuous expansion columns define a plurality of cells, wherein the side branch cell has a different shape than the plurality of cells, the framework having a proximal end and a distal end and a length defined by the distance between the proximal and distal ends of the framework and being positioned between the proximal end and the distal end of the primary tubular body.

8. The stent of claim 7, wherein the plurality of discontinuous expansion columns comprise,
a first expansion column, the first expansion column being discontinuous and having a first end and a second end, wherein the first end and second end of the first expansion column are opposingly connected to the framework, and
a second expansion column, wherein the second expansion column is longitudinally adjacent to the first expansion column, the second expansion column being discontinuous and having a first end and a second end, wherein the first end and second end of the second expansion column are opposingly connected to the framework,
wherein the distance between the first end and the second of the first expansion column and the distance between the first end and the second end of the second expansion column are equal to or less than ⅓ of the length of the framework.

9. The stent of claim 8, wherein the first ends of the first and second expansion columns are linearly aligned parallel with the axis of the primary tubular body.

10. The stent of claim 9, wherein the second ends of the first and second expansion columns are linearly aligned parallel with the axis of the primary tubular body.

11. The stent of claim 8, the plurality of discontinuous expansion columns further comprising a third expansion column, wherein the third expansion column is longitudinally adjacent to the first or second expansion column, the third expansion column being discontinuous and having a first end and a second end, wherein the first end and second end of the third expansion column are opposingly connected to the framework and wherein the distance between the first end and the second of the third expansion column is equal to or less than ¼ of the length of the framework.

12. The stent of claim 11, wherein the first ends of the first, second and third expansion columns are linearly aligned parallel with the axis of the primary tubular body.

13. The stent of claim 12, wherein the second ends of the first, second and third expansion columns are linearly aligned parallel with the axis of the primary tubular body.

14. The stent of claim 11, the primary tubular body further comprising a fourth expansion column, wherein the fourth expansion column is circumferentially oriented relative to the axis of the primary tubular body and wherein the first and second expansion columns are longitudinally between the third and fourth expansion columns, the fourth expansion column being discontinuous and having a first end and a second end, wherein the first end and second end of the fourth expansion column are opposingly connected to the framework and wherein the distance between the first end and the second of the fourth expansion column is equal to or less than ¼ of the length of the framework.

15. The stent of claim 14, wherein the first ends of the first, second, third and fourth expansion columns are linearly aligned parallel with the axis of the primary tubular body.

16. The stent of claim 15, wherein the second ends of the first, second third and fourth expansion columns are linearly aligned parallel with the axis of the tubular body.

17. The stent of claim 11, the framework having an axis extending through the proximal and distal ends of the framework, wherein the framework comprises a plurality of finger-like projections circumferentially extending between adjacent discontinuous and continuous expansion columns from the axis of the framework around the axis of the primary tubular body.

18. The stent of claim 17, the plurality of finger-like projections comprising at least four finger-like projections, wherein two of the four finger-like projections extend in a first direction and the remaining two of the four finger-like projections extend in a direction opposite to that of the first direction.

19. The stent of claim 18, wherein the four finger-like projections have bulbous terminating ends.

20. The stent of claim 17, the plurality of finger-like projections comprising at least six finger-like projections, wherein three of the six finger-like projections extend in a first direction and the remaining three of the six finger-like projections extend in a direction opposite to that of the first direction.

21. The stent of claim 14, wherein the first, second, third and fourth expansion columns are circumferentially parallel with one another.

22. The stent of claim 14, wherein the proximal end of the framework is proximal to the first, second, third and fourth expansion columns and the distal end of the framework is distal to the first, second, third and fourth expansion columns.

23. The stent of claim 14, wherein there are at least two continuous expansion columns proximal to the proximal end of the framework and at least two continuous expansion columns distal to the distal end of the framework.

24. The stent of claim 1, each of the expansion columns of the plurality of continuous expansion columns comprising a serpentine band having a band length, an amplitude and a frequency, the serpentine band having an axis that extends around the axis of the primary tubular body, and each of the expansion columns of the plurality of discontinuous expansion columns comprising a serpentine band having a band length, an amplitude and a frequency, the serpentine band having an axis that extends around the axis of the primary tubular body, wherein the band length of the serpentine bands of the plurality of continuous expansion columns is less than the band length of the serpentine bands of the plurality of discontinuous expansion columns.

25. The stent of claim 24, wherein the amplitude and/or frequency of the serpentine bands of the plurality of discontinuous expansion columns vary.

26. A stent, the stent comprising:
a main body, the main body being a tubular structure and comprising a proximal end, a distal end and an axis extending through the proximal and distal ends of the main body, the main body further comprising
a plurality of interconnected expansion columns, wherein the plurality of interconnected expansion columns are longitudinally spaced from the proximal end of the main body to the distal end of the main body and wherein the plurality of interconnected expansion columns define a plurality of cells,
the plurality of interconnected expansion columns comprising a first expansion column, wherein the first expansion column is circumferentially oriented relative to the axis of the main body, the first expansion column having a first end and a second end, and a second expansion column, wherein the second expansion column is circumferentially oriented relative to the axis of the main body and is longitudinally adjacent to the first expansion column, the second expansion column having a first end and a second end, and
a side branch, the side branch having an axis and extending radially from the main body between the proximal and distal ends of the main body, wherein the side branch comprises an expanded framework, the expanded framework being the terminating end of the side branch and having a ring shaped configuration and being positioned radially above the main body, the side branch further comprising a side branch wall positioned between the main body and the expanded framework, wherein the first and second ends of the first and second expansion columns are connected to the expanded framework and form at least part of the side branch wall.

27. The stent of claim 26, wherein the first and second ends of the first and second expansion columns are equally spaced around the expanded framework.

28. The stent of claim 26, the main body further comprising a third expansion column, wherein the third expansion column is circumferentially oriented relative to the axis of the main body and is longitudinally adjacent to the first or second expansion column, the third expansion column having a first end and a second end, wherein the first end and second end of the third expansion column are connected to the expanded framework and form at least a portion of the side branch wall.

29. The stent of claim 28, the main body further comprising a fourth expansion column, wherein the fourth expansion column is circumferentially oriented relative to the axis of the main body and wherein the first and second expansion columns are longitudinally between the third and fourth expansion columns, the fourth expansion column having a first end and a second end, wherein the first end and second end of the fourth expansion column are connected to the expanded framework and form at least a portion of the side branch wall.

30. The stent of claim 29, wherein the first and second ends of the first, second, third and fourth expansion columns are equally spaced around the expanded framework.

31. The stent of claim 29, wherein the first, second, third and fourth expansion columns are circumferentially parallel with one another.

32. The stent of claim 29, wherein the third expansion column is proximal to the fourth expansion column and there are at least two expansion columns proximal to the third expansion column and at least two expansion columns distal to the fourth expansion column.

33. A method of delivering a stent to a bifurcation site comprising the steps of:
providing a stent delivery system, the stent delivery system comprising:
a stent delivery catheter, and
a stent according to claim 1,
advancing the stent delivery system to a bifurcation of vessels; and
deploying the stent assembly at the bifurcation.
